(19) United States
(12) Patent Application Publication
(10) Pub. No.: US 2015/0151078 A1
(43) Pub. Date: Jun. 4, 2015

(54) Catheter

(71) Applicant: Somsak Bhitiyakul, Kingston, NY (US)

(72) Inventor: Somsak Bhitiyakul, Kingston, NY (US)

(21) Appl. No.: 14/093,611

(22) Filed: Dec. 2, 2013

(51) Int. Cl. A61M 25/01; A61M 25/06

(52) U.S. Cl. CPC A61M 25/0102; A61M 25/0693; A61M 2210/12

(57) ABSTRACT

Novel catheters are disclosed herein having multiple body components operatively configured to slideably and sealably interact with one another.

### RELATED FILINGS

This is the first filing made with the USPTO by the applicant regarding the present disclosure. It is related to U.S. Pat. No. 6,042,566 titled “Intravenous Injection Apparatus” filed on Nov. 2, 1999, with which it shares inventorship.

One example of how this disclosure differs from the Intravenous Injection Apparatus disclosure is in that the hollow needle of the 566 patent has herein been replaced by a solid stylet, thereby allowing a number of clinically advantageous structural changes to be made to the original design. Consequently, the Intravenous Injection Apparatus patent is hereby incorporated by reference herein in its entirety.

### BACKGROUND/FIELD

The subject matter of the present disclosure pertains to means for conducting body treating material into or out of a body or to or from the external membrane tissue surface of a body to treat said body.

The subject matter further comprising devices wherein a body treating material is placed into or removed from a body opening, or is placed under the external membraneous tissue covering the body or removed therefrom other than by being applied to the skin and permeating therethrough.

The subject matter of the application further comprising devices wherein the body treating material is placed in or removed from the body by (a) a tubular conduit having a bore or lumen hole extending therethrough, (b) transfer to or from a retaining means located on an implement placed in the body, or (c) by release or collection from a storage container surgically implanted in the body.

With the subject matter of the present disclosure further comprising devices including a body piercer, obturator rod, or stylet axially movable within body entering conduit while latter is disposed in body:

### SUMMARY

A method and device for attaining fluidic access to a vein or artery is disclosed herein, the method comprising the steps of providing a device comprised of an elongated, substantially hollow proximal-end body having an annular cross-section, a substantially hollow distal-end body having an annular cross-section removably coupled to the distal end thereof with a compliant sealing membrane therebetween and having a substantially transparent or substantially translucent cannula extending distally therefrom, and a stylet slideably coupled to the distal-end and proximal-end bodies, with the stylet being an elongated member having a solid, narrow protrusion extending distally therefrom and terminating at the distal end thereof in a pointed tip and an enlarged body at the proximal end thereof, wherein the cross-section of the protrusion is sized and shaped to sealably engage against the interior surface of the cannula, further wherein there is a first position along the travel of the stylet relative to the proximal-end and distal-end bodies in which it extends distally from the end of the cannula and a second position along the travel of the stylet wherein it is entirely disposed within the proximal-end body and in such a position separable—together with the proximal-end body from the distal-end body in a first state in which the proximal-end body, distal-end body, and stylet are connected with the tip of the stylet extending distally from the tip of the cannula, manually inserting the assembly into the vasculature of a patient, translating the enlarged proximal-end portion of the stylet proximally relative to the remainder of the assembly such that back-pressure is created within the cannular which is in turn filled by blood visible through the transparent or translucent cannula to indicate successful placement into the vasculature of a patient, translating the stylet to the proximal-most portion of its travel within the proximal-end body, and separating the stylet and proximal-end body from the distal-end body such that the membrane sealably closes thereby preventing release of blood.

According to further embodiments of the present disclosure, the device further comprises a substantially transparent or substantially translucent chamber disposed upon the proximal-end portion of the distal-end body.

According to further embodiments of the present disclosure, the device further comprises complementary bayonet mounts disposed upon the proximal-end body and distal-end body operatively configured to releasably engage each upon the other.

According to further embodiments of the present disclosure, the device further comprises complementary press-fittings disposed between proximal-end body and distal-end body operatively configured to releasably engage each upon the other.

According to further embodiments of the present disclosure, the device further comprises a port operatively configured to fluidically connect the interior volume of the device to an external reservoir extending medially form the distal end portion of the proximal-end body.

According to further embodiments of the present disclosure, the device further comprises tabs extending medially from the interior surface of the proximal end body that travel within complementary channels in the stylet thereby defining the proximal and distal limits of the motion of the stylet within the proximal-end body.

### BRIEF DESCRIPTION OF THE FIGURES

In the figures, which are not necessarily drawn to scale, like numerals describe substantially similar components throughout the several views. The drawings illustrate generally, by way of example, but not by way of limitation, various embodiments discussed in the claims of the present document.

FIG. 1 shows an isometric view of a first embodiment of a Catheter in a first condition with its stylet translated to the proximal end of its movement.

FIG. 2 shows an isometric view of an embodiment of a stylet for a Catheter.

FIG. 3 shows an isometric view of an embodiment of a proximal-end portion of a Catheter.

FIG. 4 shows a cut-away plan isometric of an embodiment of a distal-end portion of a Catheter.

FIG. 5 shows an isometric view of a first embodiment of a Catheter in a second condition with its stylet translated to the distal end of its movement and such assembly separated from a distal-end body.

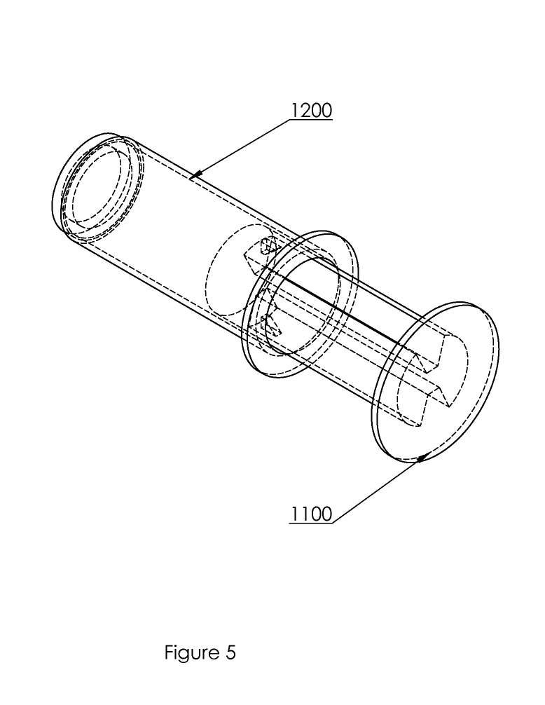

### DETAILED DESCRIPTION OF THE FIGURES

Various embodiments of the presently disclosed apparatus will now be described in detail with reference to the drawings, wherein like reference numerals identify similar or identical elements. Where numerals are used to describe elements, such signals a repeating of substantially similar structures.

In the drawings and in the description that follows, the term “proximal,” will refer to the end of a device or system that is closest to the operator, while the term “distal” will refer to the end of the device or system that is farthest from the operator. Similar, anatomical terms of reference such as dorsal, ventral, medial, and lateral shall have their accepted meanings in the arts.

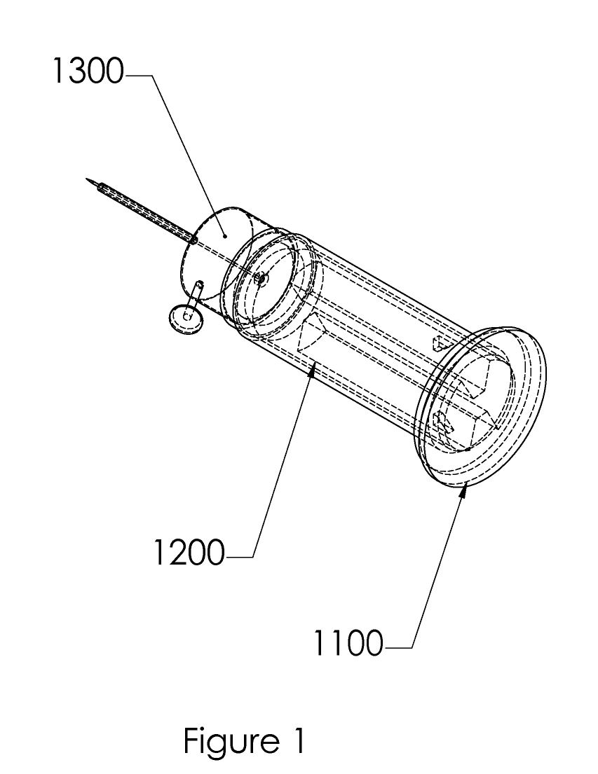

According to a first embodiment 1000 of the present disclosure shown in FIG. 1, a catheter comprises a stylet 1100, a proximal-end body 1200, and a distal-end body 1300.

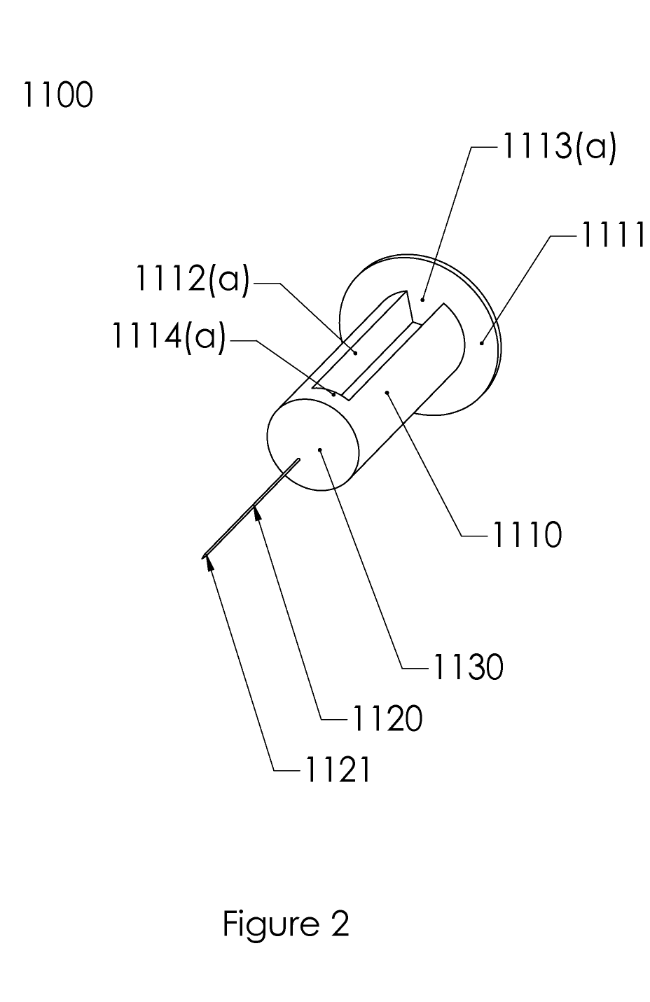

Referring now to FIG. 2, stylet comprises a substantially rigid, elongated body 1110, having an enlarged portion 1111 disposed upon the proximal-end thereof sized and shaped to be depressed by the thumb of an operator. Further, there is a pair of longitudinal grooves 1112(a and b) [1112(b) not shown] disposed upon opposing sides of body 1110 and comprising material removed therefrom. Grooves 1112(a and b) terminate at their respective distal and proximal ends with faces 1114(a and b) and 1113(a and b). There is a solid, thin, and elongated protrusion 1120 extending distally from the distal-end portion of body 1110, with 1120 terminating at a sharp point 1121 at the distal-most portion thereof.

With continued reference to FIG. 2, there is a substantially conical intermediate portion 1130 disposed between body 1110 and protrusion 1120.

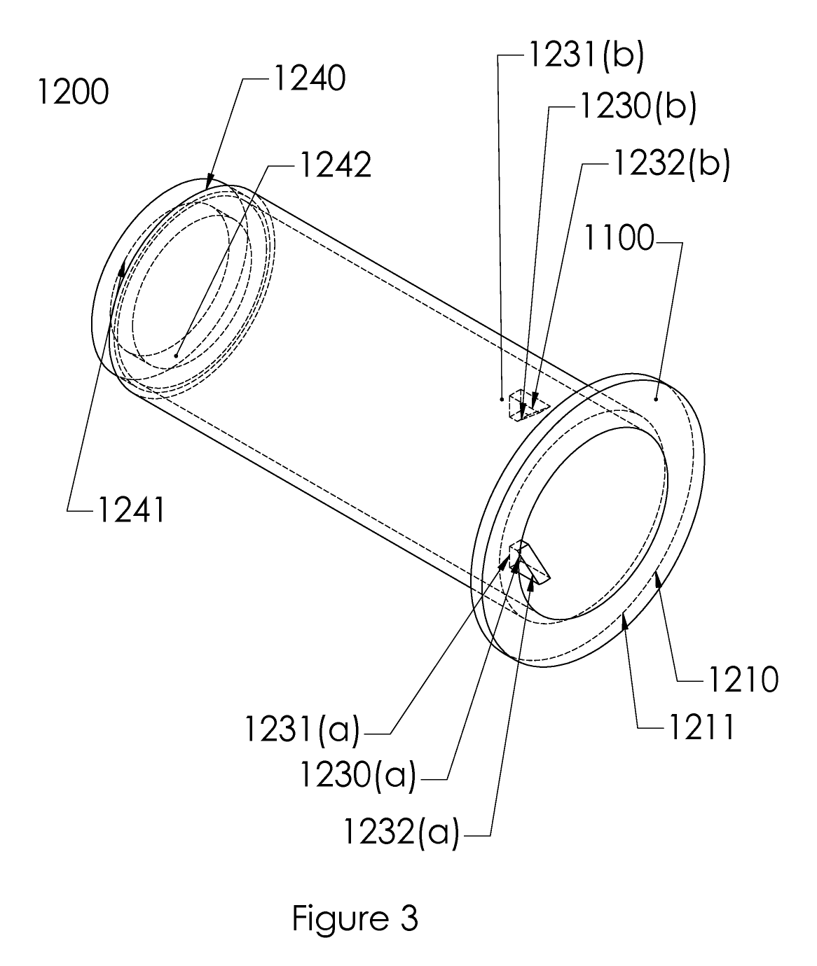

Referring now to FIG. 3, a proximal-end body 1200 is shown, with the body comprising an elongated tube 1220 with an enlarged portion 1210 at the proximal end thereof and a narrowed portion 1240 at the distal end thereof. Enlarged portion 1210 has a surface at the proximal end thereof sized and shaped to be abutted by the distal face of enlarged portion 1111 of stylet 1100. There are also corresponding protrusions 1230 (a and b) extending medially from the interior face of body 1200 sized and shaped to be slideably retained within 1112(a and b). Protrusions 1230 (a and b) each have a distal face 1231(a and b) and a proximal face 1232(a and b) disposed thereupon which interfere with corresponding proximal and distal faces 1114(a and b) and 1113(a and b) disposed upon channels 1112(a and b) thereby defining the limits of the travel of stylet 1100 relative to proximal-end portion 1200.

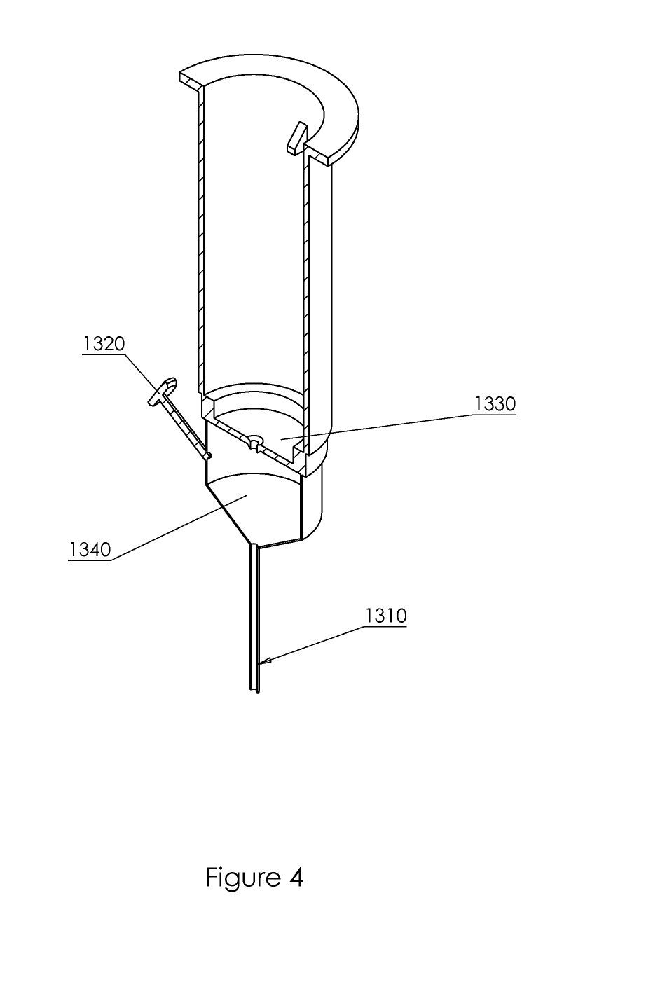

Referring now to FIG. 4, a distal-end body is shown having a substantially rigid cannula 1310, a reservoir body 1340, an auxiliary port 1320, and compliant membrane 1330.

With continued reference to FIG. 4, there is a cannula 1310 disposed upon the proximal-end of the distal-end body. Cannula 1310 is comprised of a substantially translucent or transparent material and having an inner cross-section sized and shaped to sealably engage about the exterior surface of protrusion 1120. There is a reservoir volume 1340 disposed proximally from the cannula. Reservoir volume 1340 is also composed of a substantially transparent or translucent material although not necessarily the same material from which cannula 1310 is manufactured.

There is a port 1320 extending laterally from reservoir volume 1340 operatively configured to fluidically couple to reservoir and cannula to an external volume. Port 1320 is configured to be engaged upon one of the standard medical fluidic connectors known in the art including for instance the Leur Connection disclosed in U.S. Pat. No. 4,639,019, which is incorporated by reference herein in it's entirety.

There is a membrane 1330 disposed upon the proximal face of distal-end body 1300 operatively configured to sealably engage intermediate portion 1130 of stylet 1100 when such is inserted therein, and close off the interior of reservoir volume 1340 when stylet 1100 is removed therefrom.

There is a connecting portion disposed upon the proximal end of distal-end body 1300 operatively configured to be releasably engaged upon a complementary structure disposed upon the distal end of proximal-end portion 1200. The connecting portion may be selected from concentric and complementary press-fitting grooves, complementary threads, or a bayonet mount as are known and practiced in the surgical arts.

An exemplary method of using a catheter will now be described. Initially, a catheter woo is provided in a first condition, as shown in FIG. 1, with the point 1121 protruding from cannula 1310. Next, an operator grasps the assembly in their fingers and inserts it into the vasculature of a patient. After insertion, stylet 1100 is translated proximally relative to the remainder of the assembly, thereby creating a back-pressure within cannula 1120. If the insertion has been successful, this backpressure will draw blood into the cannula and eventually reservoir volume 1340, in either of which an operator may observe such blood as evidence of a successful insertion. Next, stylet 1100 is translated to the proximal end of its travel such that protrusion 1120 is substantially retained within proximal-end body 1200. At this stage, the combined assembly of the stylet and proximal-end body may be separated from the distal-end portion, thereby sealing membrane 1330.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as embodiments.

Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A device for introducing a needle into the vasculature of a patient comprising;
an elongated, substantially hollow proximal-end body having an annular cross-section, a substantially hollow distal-end body having an annular cross-section removably coupled to the distal end thereof with a compliant sealing membrane therebetween and having a substantially transparent or substantially translucent cannula extending distally therefrom, and a stylet slideably coupled to the distal-end and proximal-end bodies, with the stylet being an elongated member having a solid, narrow protrusion extending distally therefrom and terminating at the distal end thereof in a pointed tip and an enlarged body sized and shaped to be grasped by the fingers of an operator at the proximal end thereof, wherein the cross-section of the protrusion is sized and shaped to sealably engage against the interior surface of the cannula, further wherein there is a first position along the travel of the stylet relative to the proximal-end and distal-end bodies in which it extends distally from the end of the cannula and a second position along the travel of the stylet wherein it is entirely disposed within the proximal-end body and in such a position separable—together with the proximal-end body from the distal-end body.

2. The device of claim 1, wherein there is a substantially transparent or substantially translucent chamber disposed upon the proximal-end portion of the distal-end body.

3. The device of claim 1, wherein there are complementary bayonet mounts disposed upon the proximal-end body and distal-end body operatively configured to releasably engage each upon the other.

4. The device of claim 1, wherein there are complementary press-fittings disposed between proximal-end body and distal-end body operatively configured to releasably engage each upon the other.

5. The device of claim 1, wherein the is a port operatively configured to fluidically connect the interior volume of the device to an external reservoir extending medially form the distal end portion of the proximal-end body.

6. The device of claim 1, wherein there are tabs extending medially from the interior surface of the proximal end body that travel within complementary channels in the stylet thereby defining the proximal and distal limits of the motion of the stylet within the proximal-end body.

7. A method of attaining fluidic access to a vein or artery, the method comprising the steps of providing a device comprised of an elongated, substantially hollow proximal-end body having an annular cross-section, a substantially hollow distal-end body having an annular cross-section removably coupled to the distal end thereof with a compliant sealing membrane therebetween and having a substantially transparent or substantially translucent cannula extending distally therefrom, and a stylet slideably coupled to the distal-end and proximal-end bodies, with the stylet being an elongated member having a solid, narrow protrusion extending distally therefrom and terminating at the distal end thereof in a pointed tip and an enlarged body at the proximal end thereof, wherein the cross-section of the protrusion is sized and shaped to sealably engage against the interior surface of the cannula, further wherein there is a first position along the travel of the stylet relative to the proximal-end and distal-end bodies in which it extends distally from the end of the cannula and a second position along the travel of the stylet wherein it is entirely disposed within the proximal-end body and in such a position separable—together with the proximal-end body from the distal-end body in a first state in which the proximal-end body, distal-end body, and stylet are connected with the tip of the stylet extending distally from the tip of the cannula, manually inserting the assembly into the vasculature of a patient, translating the enlarged proximal-end portion of the stylet proximally relative to the remainder of the assembly such that back-pressure is created within the cannular which is in turn filled by blood visible through the transparent or translucent cannula to indicate successful placement into the vasculature of a patient, translating the stylet to the proximal-most portion of its travel within the proximal-end body, and separating the stylet and proximal-end body from the distal-end body such that the membrane sealably closes thereby preventing release of blood.

8. The method of claim 7, wherein the device further comprises a substantially transparent or substantially translucent chamber disposed upon the proximal-end portion of the distal-end body.

9. The method of claim 7, wherein the device further comprises complementary bayonet mounts disposed upon the proximal-end body and distal-end body operatively configured to releasably engage each upon the other.

10. The method of claim 7, wherein the device further comprises complementary press-fittings disposed between proximal-end body and distal-end body operatively configured to releasably engage each upon the other.

11. The method of claim 7, wherein the device further comprises a port operatively configured to fluidically connect the interior volume of the device to an external reservoir extending medially form the distal end portion of the proximal-end body.

12. The method of claim 7, wherein the device further comprises tabs extending medially from the interior surface of the proximal end body that travel within complementary channels in the stylet thereby defining the proximal and distal limits of the motion of the stylet within the proximal-end body.
